(19) United States
(12) Patent Application Publication
(10) Pub. No.: US 2021/0225491 A1
(43) Pub. Date: Jul. 22, 2021

(54) DIAGNOSTIC IMAGE CONVERTING APPARATUS, DIAGNOSTIC IMAGE CONVERTING MODULE GENERATING APPARATUS, DIAGNOSTIC IMAGE RECORDING APPARATUS, DIAGNOSTIC IMAGE CONVERTING METHOD, DIAGNOSTIC IMAGE CONVERTING MODULE GENERATING METHOD, DIAGNOSTIC IMAGE RECORDING METHOD, AND COMPUTER RECORDABLE RECORDING MEDIUM

(71) Applicant: Young Saem AHN, Gunpo-si, Gyeonggi-do (KR)

(72) Inventors: Chengbin JIN, Incheon (KR); Weon Jin KIM, Seoul (KR); Eun Sik PARK, Seoul (KR); Yeong Saem AHN, Gunpo-si, Gyeonggi-do (KR); Bin YANG, Incheon (KR); Mingjie LIU, Incheon (KR); Seong Su JOO, Seoul (KR)

(21) Appl. No.: 16/304,477

(22) PCT Filed: Nov. 16, 2018
(86) PCT No.: PCT/KR2018/014151

§ 371 Date: Nov. 26, 2018

(30) Foreign Application Priority Data
Nov. 17, 2017 (KR) 10-2017-0154251
Nov. 16, 2018 (KR) 10-2018-0141923

(51) Int. Cl. G16H 30/40; G06T 7/00; G06T 3/00; G06T 3/40

(52) U.S. Cl. CPC G16H 30/40; G06T 7/0012; G06T 3/0075; G06T 3/4046; G06T 2207/30016; G06T 2207/10088; G06T 2207/10116; G06T 2207/20081; G06T 2207/20084; G06T 2207/10081

(57) ABSTRACT

An apparatus for converting a diagnostic image according to some embodiments of the present invention includes an input unit for inputting a CT image, a converting module configured to convert the CT image inputted via the input unit into an MRI image, and an output unit configured to output the MRI image converted by the converting module.

### TECHNICAL FIELD

The present invention relates to a diagnostic image converting apparatus, a diagnostic-image-converting-module generating apparatus, a diagnostic image recording apparatus, a diagnostic image converting method, a diagnostic-image-converting-module generating method, a diagnostic image recording method, and a computer readable recording medium.

### BACKGROUND

Diagnostic imaging technology is a medical technology for imaging the human body structure and anatomical images by using ultrasound, Computerized Tomography (CT), and Magnetic Resonance Imaging (MRI). Thanks to the development of artificial intelligence, automated analysis of medical images using such diagnostic imaging techniques has become possible up to a practical level for actual medical care.

Korean Patent Application Publication No. 2017-0085756 discloses a combined MRI and CT or MRCT diagnostic device that combines a CT apparatus and an MRI apparatus so that the CT apparatus rotates its signal source into a transformed signal source of magnetic field signals of the MRI apparatus.

CT scans are used in emergency rooms and the like to provide detailed information on the structure of the bone, while MRI apparatuses are suitable for soft tissue examination and tumor detection, etc. in case of ligament and tendon injuries.

CT apparatus is advantageous that it can obtain a clear image by using x-ray with minimized motion artifact due to its short scanning time. A CT scan with an intravenous contrast agent provides a CT angiogram when the scanning is performed at the highest concentration of the agent in the blood vessel.

MRI apparatus detects anatomical changes of the human body by using the principle of nuclear magnetic resonance, and it can obtain high-resolution anatomical images without exposing the body to radiation. CT scan can show only cross-sectional images, whereas MRI allows one to view the affected part with stereoscopic images showing all the longitudinal and lateral cross sections to carry out finer inspection with images with higher resolution than that of CT.

The CT scan needs only several minutes to complete its inspection, but the MRI scan takes about 30 minutes to an hour. Therefore, in an emergency, such as a traffic accident or a cerebral hemorrhage, a CT with short examination time is useful.

MRI has the advantage of presenting more precise three-dimensional images than CT, which can be viewed from various angles. MRI enables a more accurate diagnosis of soft tissues such as muscles, cartilage, ligaments, blood vessels, and nerves compared to CT.

On the other hand, patients with cardiac pacemakers, metal implants, or tattoos, are prohibited from using MRI for reasons such as patient risk of injury and image distortion (shaking or noise).

### PRIOR ART DOCUMENT

### Patent Literature

Patent Document 1: Korean Patent Application Publication No. 2017-0085756

### DISCLOSURE

### Technical Problem

In an emergency, such as a traffic accident or a cerebral hemorrhage, a CT is useful for its shorter examination time, but there are diseases that are difficult to see through CT. MRI has a slower examination time, but can tell more than CT. Therefore, a CT image alone that is provided with the equivalent effect to that of an MRI image could not only save more lives in an emergency situation, but also save the time and cost otherwise required for MRI imaging.

One aspect of the present invention, seeking to address the above deficiencies, provides a diagnostic image converting apparatus for obtaining an MRI image from a CT image.

It is another object of the present invention to provide an apparatus for generating a diagnostic image converting module for obtaining an MRI image from a CT image.

It is yet another object of the present invention to provide a diagnostic image recording apparatus for obtaining an MRI image from a CT image.

It is yet another object of the present invention to provide a diagnostic image converting method for obtaining an MRI image from a CT image.

It is yet another object of the present invention to provide a method of generating a diagnostic image converting module for obtaining an MRI image from a CT image.

It is yet another object of the present invention to provide a diagnostic image recording method for obtaining an MRI image from a CT image.

The technical challenge of the present invention is not limited to those mentioned above, and other unmentioned challenges will be clearly understandable to those of ordinary skill in the art from the following description.

### SUMMARY

According to some embodiments of the present invention, an apparatus for converting a diagnostic image includes an input unit for inputting a CT image, a converting module configured to convert the CT image inputted via the input unit into an MRI image, and an output unit configured to output the MRI image converted by the converting module.

According to some embodiments of the present invention, the apparatus further includes a classifying unit configured to classify the CT image inputted via the input unit by positions of recorded tomographic layers. The converting module is configured to convert the CT image classified by the classifying unit into the MRI image.

According to some embodiments of the present invention, the classifying unit is configured, by the positions of the recorded tomographic layers, to classify an image of from a top of a brain to right before an eyeball appears as a first layer image, to classify an image of from the eyeball beginning to appear to right before a lateral ventricle appears as a second layer image, to classify an image of from the lateral ventricle beginning to appear to right before a ventricle disappears as a third layer image, and to classify an image of from the ventricle disappears to a bottom of the brain as a fourth layer image.

According to some embodiments of the present invention, the converting module includes a first converting module configured to convert a CT image classified as the first layer image into the MRI image, a second converting module configured to convert a CT image classified as the second layer image into the MRI image, a third converting module configured to convert a CT image classified as the third layer image into the MRI image, and a fourth converting module configured to convert a CT image classified as the fourth layer image into the MRI image.

According to some embodiments of the present invention, the apparatus further includes a pre-processing unit configured to perform a pre-processing including at least one of normalization, gray scaling, or resizing on the CT image inputted via the input unit.

According to some embodiments of the present invention, the apparatus further includes a post-processing unit configured to perform a post-processing including a deconvolution on the MRI image converted by the converting module.

According to some embodiments of the present invention, the apparatus further includes an evaluation unit configured to output a first likelihood that the MRI image converted by the converting module is a CT image and a second likelihood that the MRI image converted by the converting module is an MRI image.

According to some embodiments of the present invention, an apparatus for generating a converting module of the apparatus for converting a diagnostic image includes an MRI generator configured, when a first CT image that is training data is inputted, to generate a first MRI image from the first CT image by performing a plurality of operations, a CT generator configured, when a second MRI image that is training data is inputted, to generate a second CT image from the second MRI image by performing a plurality of operations, an MRI discriminator configured, when the first MRI image and the second MRI image are inputted, to output a first likelihood of the input image being an MRI image and a second likelihood of the input image not being an MRI image by performing a plurality of operations, a CT discriminator configured, when the first CT image and the second CT image are inputted, to output a third likelihood of the input image being a CT image and a fourth likelihood of the CT image not being a CT image by perform a plurality of operations, an MRI likelihood loss estimator configured to calculate a first likelihood loss that is a difference between an expected value and an output value of the first likelihood and the second likelihood outputted from the MRI discriminator, a CT likelihood loss estimator configured to calculate a second likelihood loss that is a difference between an expected value and an output value of the third likelihood and the fourth likelihood outputted from the CT discriminator, an MRI reference loss estimator configured to calculate a first reference loss that is a difference between the first MRI image and the second MRI image, and a CT reference loss estimator configured to calculate a second reference loss that is a difference between the first CT image and the second CT image. The apparatus is configured to adjust weights included in the plurality of operations performed by the MRI generator, the CT generator, the MRI discriminator, and the CT discriminator using a back propagation algorithm, in order to minimize the first and second likelihood losses and the first and second reference losses.

According to some embodiments of the present invention, the apparatus is configured to adjust the weights by using paired data and unpaired data.

According to some embodiments of the present invention, An apparatus for recording a diagnostic image includes an X-ray generator configured to generate X-rays for CT imaging, a data acquisition unit configured to detect the X-rays generated by the X-ray generator and penetrated through a human body, to convert detected X-rays into electrical signals, and to acquire image data from converted electrical signals, an image construction unit configured to construct a CT image from the image data acquired by the data acquisition unit and to output the CT image, an apparatus for converting a diagnostic image configured to receive the CT image constructed by the image construction unit, to convert the CT image into an MRI image, and to output the MRI image, and a display unit configured to display the CT image and the MRI image selectively or concurrently.

According to some embodiments of the present invention, a method of converting a diagnostic image includes inputting a CT image, converting the CT image inputted at the inputting into an MRI image, and outputting the MRI image converted at the converting.

According to some embodiments of the present invention, the method further includes classifying the CT image inputted at the inputting by positions of recorded tomographic layers. The converting includes converting the CT image classified at the classifying into the MRI image.

According to some embodiments of the present invention, the classifying includes, by the positions of the recorded tomographic layers, classifying an image of from a top of a brain to right before an eyeball appears as a first layer image, classifying an image of from the eyeball beginning to appear to right before a lateral ventricle appears as a second layer image, classifying an image of from the lateral ventricle beginning to appear to right before a ventricle disappears as a third layer image, and classifying an image of from the ventricle disappears to a bottom of the brain as a fourth layer image.

According to some embodiments of the present invention, the converting includes first converting including converting a CT image classified as the first layer image into the MRI image, second converting including converting a CT image classified as the second layer image into the MRI image, third converting including converting a CT image classified as the third layer image into the MRI image, and fourth converting including converting a CT image classified as the fourth layer image into the MRI image.

According to some embodiments of the present invention, the method further includes performing a pre-processing including at least one of normalization, gray scaling, or resizing on the CT image inputted at the inputting.

According to some embodiments of the present invention, the method further includes performing a post-processing including a deconvolution on the MRI image converted at the converting.

According to some embodiments of the present invention, the method further includes outputting a first likelihood that the MRI image converted at the converting is a CT image and a second likelihood that the MRI image converted at the converting module is an MRI image.

According to some embodiments of the present invention, a method of generating a converting module used at the converting in the method of converting a diagnostic image includes first generating including generating, when a first CT image that is training data is inputted, a first MRI image from the first CT image by performing a plurality of operations, second generating including generating, when a second MRI image that is training data is inputted, a second CT image from the second MRI image by performing a plurality of operations, first outputting including outputting, when the first MRI image and the second MRI image are inputted, a first likelihood of the input image being an MRI image and a second likelihood of the input image not being an MRI image by performing a plurality of operations, second outputting including outputting, when the first CT image and the second CT image are inputted, a third likelihood of the input image being a CT image and a fourth likelihood of the CT image not being a CT image by perform a plurality of operations, calculating a first likelihood loss that is a difference between an expected value and an output value of the first likelihood and the second likelihood outputted at the first outputting, calculating a second likelihood loss that is a difference between an expected value and an output value of the third likelihood and the fourth likelihood outputted at the second outputting, calculating a first reference loss that is a difference between the first MRI image and the second MRI image, calculating a second reference loss that is a difference between the first CT image and the second CT image, and adjusting weights included in the plurality of operations performed at the first generating, the second generating, the first outputting, and the second outputting using a back propagation algorithm, in order to minimize the first and second likelihood losses and the first and second reference losses.

According to some embodiments of the present invention, the adjusting includes adjusting the weights by using paired data and unpaired data.

According to some embodiments of the present invention, a method of recording diagnostic image includes generating X-rays for CT imaging, acquiring including detecting the X-rays generated at the generating and penetrated through a human body, converting detected X-rays into electrical signals, and acquiring image data from converted electrical signals, first outputting including constructing a CT image from the image data acquired at the acquiring and outputting the CT image, converting including performing the method of converting a diagnostic image according to any one of claims 1 to 7, by receiving the CT image constructed at the constructing, converting the CT image into an MRI image, and outputting the MRI image, and displaying the CT image and the MRI image selectively or concurrently.

According to some embodiments of the present invention, a non-transitory computer readable recording medium stores a computer program including computer-executable instructions for causing, when executed by a processor, the processor to perform the method of converting a diagnostic image according to some embodiments of the present invention.

According to some embodiments of the present invention, a non-transitory computer readable recording medium stores a computer program including computer-executable instructions for causing, when executed by a processor, the processor to perform the method of generating a converting module according to some embodiments of the present invention.

According to some embodiments of the present invention, a non-transitory computer readable recording medium stores a computer program including computer-executable instructions for causing, when executed by a processor, the processor to perform the method of recording a diagnostic image according to some embodiments of the present invention.

### Advantageous Effects

As described above, at least one embodiment of the present invention is effective to provide a diagnostic image converting apparatus for obtaining an MRI image from a CT image.

At least one embodiment of the present invention is effective to provide an apparatus for generating a diagnostic image converting module for obtaining an MRI image from a CT image.

At least one embodiment of the present invention is effective to provide a diagnostic image recording apparatus for obtaining an MRI image from a CT image.

At least one embodiment of the present invention is effective to provide an diagnostic image converting method of obtaining an MRI image from a CT image.

At least one embodiment of the present invention is effective to provide a method of generating a diagnostic image converting module for obtaining an MRI image from a CT image.

At least one embodiment of the present invention is effective to provide a diagnostic image recording method for obtaining an MRI image from a CT image.

At least one embodiment of the present invention, by converting the CT image in MRI imaging, as well as to obtain more of the life in emergency situations, there is an effect that it is possible to save time and cost required for the MRI scans.

The effect of the invention is not limited to those mentioned above, and other unmentioned effects will be clearly understandable to those of ordinary skill in the art from the following description.

### BRIEF DESCRIPTION OF THE DRAWINGS

The accompanying drawings are same as the accompanying drawings of Korean Pat. Appl. No. 10-2017-0154251 and Korean Pat. Appl. No. 10-2018-0141923 upon which the present PCT application is based and from which the present PCT application claims the benefit of priority.

FIG. 1 is images illustrating paired data and unpaired data used by the diagnostic image converting apparatus according to at least one embodiment of the present invention.

FIG. 2 is a functional block diagram of a diagnostic image converting apparatus according to at least one embodiment of the present invention.

FIGS. 3A to 3D are example images classified by a classifying unit of a diagnostic image converting apparatus according to at least one embodiment of the present invention.

FIG. 4 is a functional block diagram of a converting unit of a diagnostic image converting apparatus according to at least one embodiment of the present invention.

FIGS. 5 and 6 are conceptual diagrams for explaining the training of a converting unit of a diagnostic image converting apparatus according to at least one embodiment of the present invention.

FIG. 7 is a flowchart of a training method of a converting unit of a diagnostic image converting apparatus according to at least one embodiment of the present invention.

FIG. 8 is a flowchart of a diagnostic image converting method according to at least one embodiment of the present invention.

FIG. 9 is images for explaining the generation of paired data between CT and MRI images.

FIGS. 10A to 10D are conceptual diagrams of an example dual cycle-consistent structure using paired data and unpaired data.

FIG. 11 is input CT images, synthesized MRI images, reference MRI images, and absolute errors between real and synthesized MRI images.

FIG. 12 is input CT images, synthesized MRI images when using paired data, unpaired data, and paired and unpaired data together, respectively, and reference MRI images.

FIG. 13 is a functional block diagram of a diagnostic image recording apparatus according to at least one embodiment of the present invention.

### DETAILED DESCRIPTION

With reference to the accompanying drawings, the following describes in detail a diagnostic image converting apparatus, diagnostic-image-converting-module generating apparatus, diagnostic image recording apparatus, diagnostic image converting method, diagnostic-image-converting-module generating method, diagnostic image recording method, and computer-readable recording media in accordance with some embodiments of the present invention.

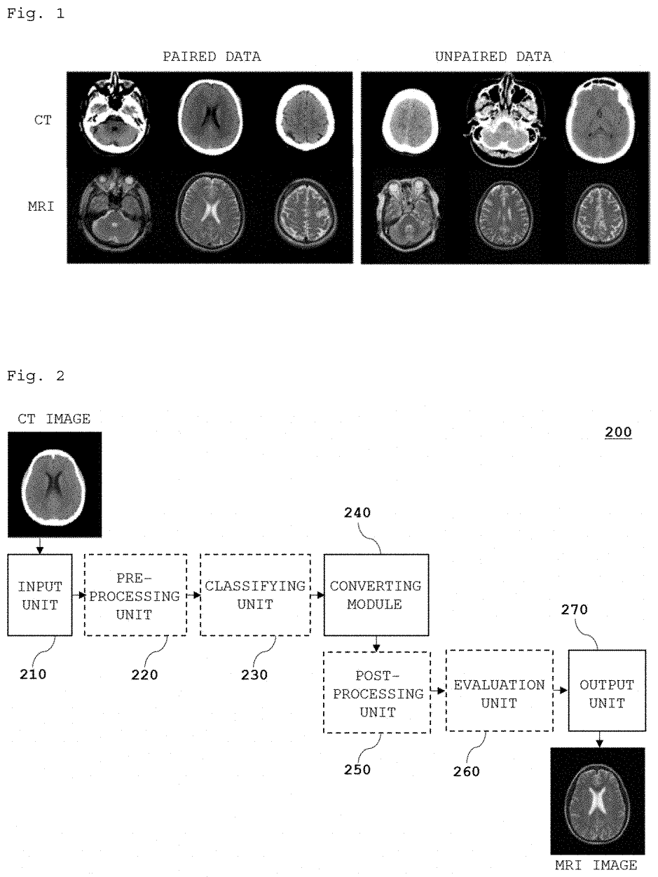

FIG. 1 is images illustrating paired data and unpaired data used by the diagnostic image converting apparatus according to at least one embodiment of the present invention.

There are publicized image translating or converting technologies including converting an MRI image to a CT image by using the pix2pix model through training with paired data, converting a CT image to a synthesized positron emission tomography (PET) image by using fully convolutional network (FCN) and a pix2pix model through training with paired data, converting a CT image to a PET image by using the pix2pix model through training with paired data, and converting an MRI image to a CT image by using a cycleGAN model through training with unpaired data.

In FIG. 1, the left side is paired data which include CT and MR slices taken from the same patient at the same anatomical location, and the right side is unpaired data which include CT and MR slices that are taken from different patients at different anatomical locations.

A paired training method using paired data results in a fair output, and needs no large numbers of aligned CT and MRI image pairs to obtain, which is advantageous. However, obtaining rigidly aligned data can be not only difficult but also expensive, which would counter the advantage of the paired training method.

Conversely, an unpaired training method using unpaired data can take advantage of a considerable amount of available data, which would increase the amount of training data exponentially, and alleviate many of the constraints of current deep learning-based synthetic systems. However, the unpaired training method has lower quality of the result and exhibits a substantially inferior performance compared to the paired training method.

Some embodiments of the present invention convert a CT image to an MRI image by using paired and unpaired data, whereby providing an approach that complements the deficiencies of the paired training method and of the unpaired training method.

FIG. 2 is a functional block diagram of a diagnostic image converting apparatus 200 according to at least one embodiment of the present invention.

As shown in FIG. 2, a diagnostic image converting apparatus 200 according to at least one embodiment of the present invention includes an input unit 210, a pre-processing unit 220, a classifying unit 230, a converting unit 240, a post-processing unit 250, an evaluation unit 260, and an output unit 270, and it converts and provides a CT image of, for example, a brain to an MRI image.

The pre-processing unit 220, upon receiving the CT image via the input unit 210, performs pre-processing of the CT image and provides the preprocessed CT image to the classifying unit 230. Here, the pre-processing includes, for example, a normalization, gray scaling, resizing and the like.

In at least one embodiment of the present invention, the pre-processing unit 220 operates as expressed by the following Equation 1, to perform the min-max normalization on the respective pixel values of the inputted CT image, and to convert the normalized pixel values to such pixel values that fall in a predetermined range.

$${v}^{′} = \frac{v - min\_a}{max\_a - min\_a} ( max\_b - min\_b ) + min\_b . \qquad [ Equation 1 ]$$

Here, v is the pixel value of the inputted CT image, v′ is a pixel value obtained by normalizing the pixel value v. In addition, min a and max a are the minimum and maximum pixel values of the inputted CT image, and min b and max b are the minimum and maximum pixel values within the range of pixel values to be normalized.

After normalization, the pre-processing unit 220 performs gray scaling for adjusting the number of image channels of the CT image to one. Then, the pre-processing unit 220 resizes the CT image into a predetermined size. For example, the pre-processing unit 220 may adjust the size of the CT image to 256×256×1.

The classifying unit 230 classifies the inputted CT image into one of a predetermined number of (e.g., four) classes. Brain CT imaging captures images of vertical cross-sections of the brain of a lying person subject to the CT scan.

According to at least one embodiment of the present invention, the brain cross-section is divided into four layers, depending on whether or not the eyeball portion belongs to them and on whether or not the lateral ventricle and ventricle belong to them. Accordingly, the classifying unit 230 classifies the CT brain images from its top to bottom into four layers, depending on whether the eye part belongs to them and on whether the lateral ventricle and ventricle belong to them.

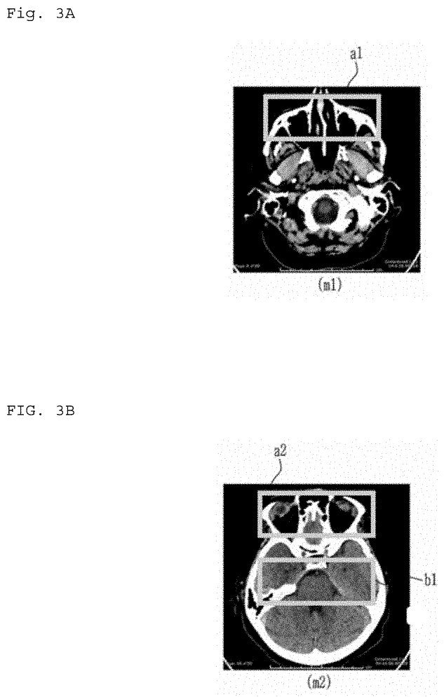

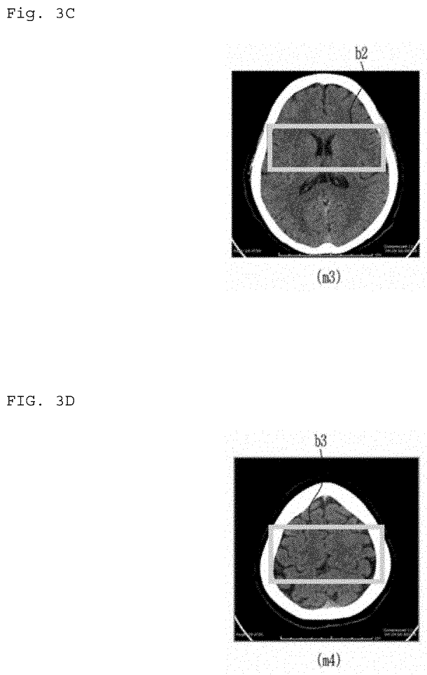

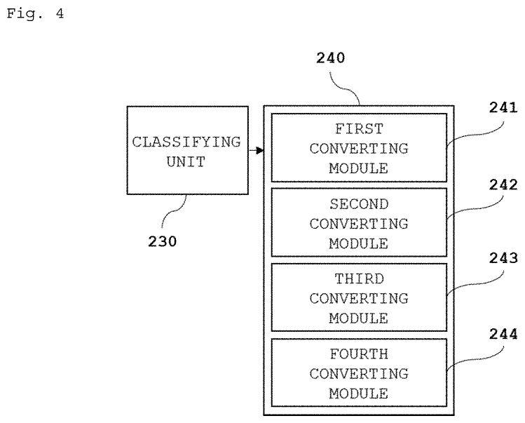

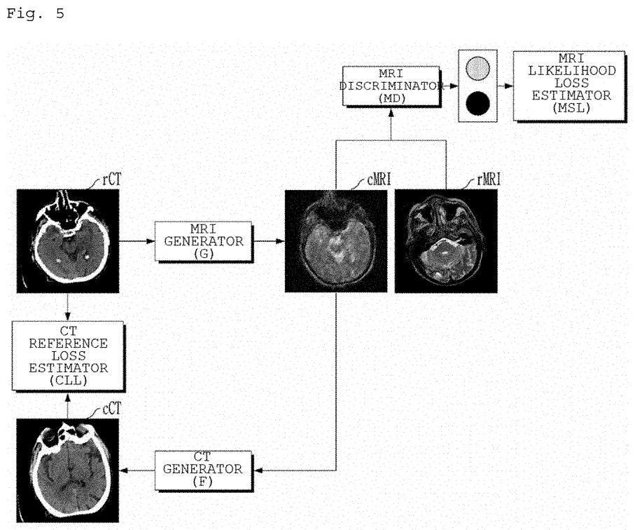

FIGS. 3A to 3D are example images classified by the classifying unit 230 of the diagnostic image converting apparatus 200 according to at least one embodiment of the present invention.

FIG. 3A illustrates a first layer image at m1. The classifying unit 230 may classify as first layer image m1, such images as taken from the top of the brain up to right before the eyeball emerges. Thus, the first layer image m1 is images taken sequentially from the top of the brain up to right before the eyeball portion of the brain shows, wherein the portion at a1 shows no eyeball portion of the brain.

FIG. 3B illustrates a second layer image at m2. The classifying unit 230 classifies as the second layer image m2, such images that range from where the eyeball emerges up to right before the lateral ventricle emerges. Since the second layer image m2 is images taken from where the eyeball emerges as visible at a2 up to right before the lateral ventricle shows as visible at b1, it includes the eyeball portion with no visible lateral ventricle.

FIG. 3C illustrates a third layer image at m3. The classifying unit 230 classifies as the third layer image m3, such images that range from where the lateral ventricle emerges up to right before the ventricle disappears. Since the third layer image m3 is images taken from where the lateral ventricle emerges up to right before the ventricle disappears, it presents the lateral ventricle or the ventricle.

FIG. 3D illustrates a fourth layer image at m4. The classifying unit 230 classifies as the fourth layer image m4, such images that range from where the ventricle disappears up to the bottom of the brain. Thus, the fourth layer image m4 is images taken from where the ventricle disappears up to the bottom of the brain, and it includes neither the lateral ventricle nor the ventricle.

Although FIGS. 3A to 3D illustrate classification of the brain section into a plurality of layers of the CT image, an MRI image also can be classified as above, as with the CT image.

The classifying unit 230 includes an artificial neural network. The artificial neural network can be a convolutional neural network (CNN). Accordingly, the classifying unit 230 can take the first to fourth layer images m1, m2, m3, and m4 as training data to learn thereof.

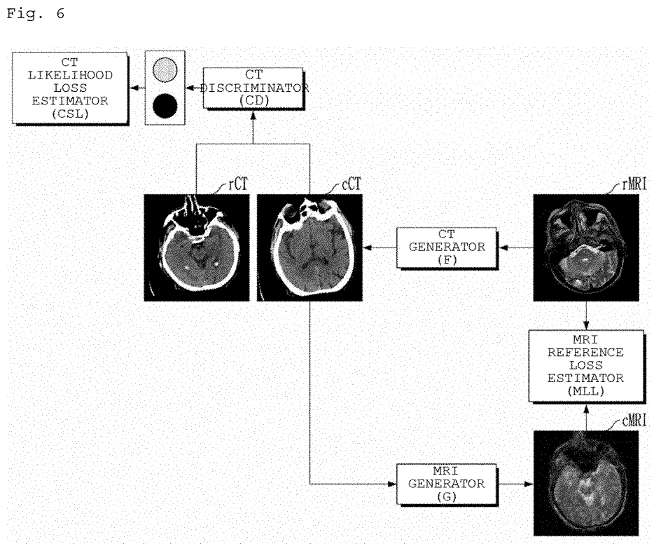

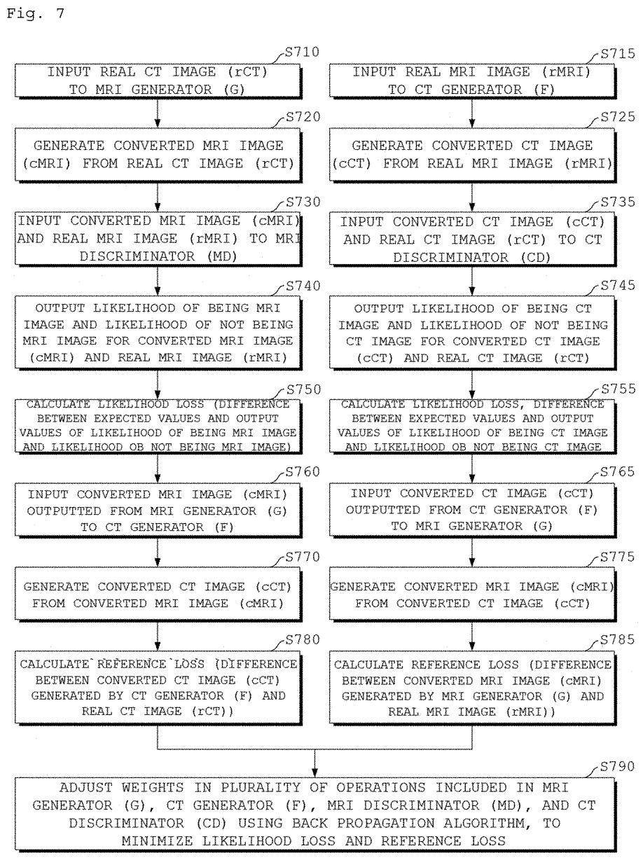

FIG. 4 is a functional block diagram of a converting unit 240 of a diagnostic image converting apparatus according to at least one embodiment of the present invention. FIGS. 5 and 6 are conceptual diagrams for explaining the training of the converting unit 240 of the diagnostic image converting apparatus 200 according to at least one embodiment of the present invention.

As shown in FIG. 4, the converting unit 240 includes first to fourth converting modules 241, 242, 243, and 244. The first to fourth converting modules 241, 242, 243, and 244 each corresponds to the first layer image to the fourth layer image m1, m2, m3, and m4. Accordingly, the classifying unit 230 classifies the input CT images as the first to fourth layer images m1, m2, m3, and m4, and then transfers the same to the relevant one of the first to fourth converting modules 241, 242, 243, and 244.

The converting unit 240 converts the CT images input from the classifying unit 230 into MRI images.

The first to fourth converting modules 241, 242, 243, and 244 each includes an artificial neural network. The artificial neural network can be generative adversarial networks (GAN). FIGS. 5 and 6 show detailed configurations of the artificial neural networks included respectively in the first to fourth converting modules 241, 242, 243, and 244 according to at least one embodiment of the present invention.

The respective artificial neural networks included in the first to fourth converting modules 241, 242, 243, and 244 includes an MRI generator G, a CT generator F, an MRI discriminator MD, a CT discriminator CD, an MRI likelihood loss estimator MSL, a CT likelihood loss estimator CSL, MRI reference loss estimator MLL, and a CT reference loss estimator CLL.

Each of the MRI generator G, CT generator F, MRI discriminator MD, and CT discriminator CD is an individual artificial neural network and can be CNN. Each of the MRI generator G, CT generator F, MRI discriminator MD, and CT discriminator CD includes a plurality of layers, each layer including a plurality of arithmetic operations. In addition, each of the plurality of arithmetic operations includes a weight.

The plurality of layers includes at least one of an input layer, a convolution layer, a polling layer, a fully-connected layer, and an output layer. The plurality of arithmetic operations includes a convolution operation, a polling operation, a Sigmode operation, a hyper tangential operation among others. Each of these operations is performed upon receiving the result of the operation of the previous layers, and each operation includes a weight.

Referring to FIGS. 5 and 6, upon receiving an input CT image, the MRI generator G performs a plurality of arithmetic operations to generate an MRI image.

Specifically, the MRI generator G performs a plurality of arithmetic operations on a pixel-by-pixel basis, and converts input CT image pixels into MRI image pixels through a plurality of arithmetic operations to generate an MRI image. The CT generator F is responsive to an input MRI image for generating a CT image by performing a plurality of arithmetic operations. Specifically, the CT generator F performs a plurality of arithmetic operations on a pixel-by-pixel basis, and converts input MRI image pixels into CT image pixels through a plurality of arithmetic operations to generate a CT image.

As shown in FIG. 5, upon receiving an input image, the MRI discriminator MD performs a plurality of arithmetic operations on the input image to output the likelihood that the input image is an MRI image and the likelihood that the input image is not an MRI image. Here, an MRI image cMRI generated by the MRI generator G or the MRI image rMRI as the training data is input as the image input to the MRI discriminator MD.

The MRI likelihood loss estimator MSL receives, from the MRI discriminator MD, its output value that is the likelihood that the input image is an MRI image and the likelihood that the input image is not an MRI image, and it calculates a likelihood loss, that is, the difference between the output value and the expected value of the likelihoods of the input image being and not being an MRI image. At this time, the softmax function may be used to calculate the likelihood loss.

The MRI discriminator MD receives the MRI image that is generated by the MRI generator G or the MRI image that is training data. When the MRI generator G is sufficiently trained, the MRI discriminator MD can expect that the MRI image generated by the MRI generator G or the MRI image, which is training data, can be both discriminated as MRI images. In that case, the MRI discriminator MD can expect such outputs that the likelihood of being the MRI image is higher than the likelihood of not being the MRI image, that the likelihood of being the MRI image is higher than a predetermined value, and that the likelihood of not being the MRI image is lower than the predetermined value. However, when the training is insufficiently performed, a difference exists between the output value and the expected value of the MRI discriminator MD, and the MRI likelihood loss estimator MSL calculates the difference between the output value and the expected value.

When the MRI generator G generates the MRI image cMRI from the CT image rCT input to the MRI generator G, the CT generator F may regenerate a CT image cCT from the generated MRI image cMRI. The CT reference loss estimator CLL calculates a reference loss which is a difference between the CT image cCT regenerated by the CT generator F and its causative CT image rCT inputted to the MRI generator G. This reference loss may be calculated by the L2 norm operation.

As shown in FIG. 6, upon receiving an input image, the CT discriminator CD performs a plurality of arithmetic operations on the input image to output the likelihood that the input image is a CT image and the likelihood of not being the CT image. Here, the CT image cCT generated by the CT generator F or the CT image rCT serving as training data is input as the input image to the CT discriminator CD.

The CT likelihood loss estimator CSL receives, from the CT discriminator CD, its output value that is the likelihood that the input image is a CT image and the likelihood of not being an CT image, and it calculates a likelihood loss, that is, the difference between the output value and the expected value of the likelihoods of the input image being and not being an CT image. Here, the softmax function may be used to calculate the likelihood loss.

The CT discriminator CD receives the MRI image that is generated by the CT generator F or the MRI image that is training data. When the CT generator F is sufficiently trained, the CT discriminator CD can expect that the CT image cCT generated by the CT generator F or the CT image rCT, which is training data, can be both discriminated as CT images. In that case, the CT discriminator CD can expect such outputs that the likelihood of being the CT image is higher than the likelihood of not being the CT image, that the likelihood of being the CT image is higher than a predetermined value, and that the likelihood of not being the CT image is lower than the predetermined value. However, when the training is insufficiently performed, a difference exists between the output value and the expected value of the CT discriminator CD, and the CT likelihood loss estimator CSL calculates the difference between the output value and the expected value.

When the CT generator F generates the MRI image cMRI from the MRI image rMRI input to the CT generator F, the MRI generator G may regenerate an MRI image cMRI from the generated CT image cCT. The MRI reference loss estimator MLL calculates a reference loss which is a difference between the MRI image cMRI regenerated by the MRI generator G and its causative MRI image rMRI inputted to the CT generator F. This reference loss may be calculated by the L2 norm operation.

Basically, the artificial neural network of the converting unit 240 is for converting a CT image into an MRI image. To this end, the MRI generator G generates, upon receiving an input CT image, an MRI image by performing a plurality of arithmetic operations. This needs deep learning for the MRI generator G. Now, description will be provided as to the training method through the aforementioned MRI generator G and the CT generator F, the MRI discriminator MD, the CT discriminator CD, the MRI likelihood loss estimator MSL, the CT likelihood loss estimator CSL, the MRI reference loss estimator MLL, and the CT reference loss estimator CLL.

The CT imaging and the MRI imaging commonly captures the cross section of the brain, but they cannot image exactly matching cross sections due to the system characteristics of CT and MRI. Therefore, it can be said that there is no MRI image that has the same section as the CT image. Therefore, in order to train how to convert CT images into MRI images, a likelihood loss and a reference loss are obtained through the forward process as shown in FIG. 5 and the backward process as shown in FIG. 6, and to minimize the likelihood loss and the reference loss, a correction is made through a back propagation to weights in the plurality of arithmetic operations included in the MRI generator G, the CT generator F, the MRI discriminator MD, and the CT discriminator CD.

The converting unit 240, which is well trained with the artificial neural network of each of the first to fourth converting modules 241, 242, 243, and 244, is operative to convert any one CT image of the first to fourth layer images m1, m2, m3, m4 when inputted, into an MRI image through an artificial neural network of a corresponding one of the first to fourth converting modules 241, 242, 243, and 244. In this manner, the converted MRI image is provided to the post-processing unit 250.

The post-processing unit 250 performs post-processing on the MRI image converted by the converting unit 240. The post-processing may be a deconvolution for improving the image quality. Here, the deconvolution may be inverse filtering, focusing, or the like. The post-processing unit 250 is optional and can be omitted if necessary.

The evaluation unit 260 outputs the likelihood that the MRI image converted by the converting unit 240 or the MRI image through the post-processing unit 250 is an MRI image and the likelihood that the MRI image is a CT image. The evaluation unit 260 includes an artificial neural network which may be CNN. The evaluation unit 260 includes at least one of an input layer, a convolution layer, a polling layer, a fully-connected layer, and an output layer, each layer including a plurality of arithmetic operations each including at least one of a polling operation, a Sigmode operation, and a hyper tangential operation. Each operation includes a weight.

The training data may be a CT image or an MRI image. When the CT image is input as training data to the artificial neural network, the output of the artificial neural network is expected to have the higher likelihood of being a CT image than the likelihood of being an MRI image. When the MRI image is input as the training data, the output of the artificial neural network is expected to have the higher likelihood of being an MRI image than the likelihood of being a CT image. During training, the expected value for this output differs from the actual output value. Therefore, after inputting the training data, the difference between the expected value and the output value is obtained, and to minimize the difference between the two values, a correction is made through the back propagation algorithm to the weights in the plurality of arithmetic operations in the artificial neural network of the evaluation unit 260.

The training is determined to be sufficiently performed when any more training data input causes the difference between the expected value and the output value to be equal to or less than a predetermined value as well as to stand still. After sufficient training is performed, the evaluation unit 260 is used to determine whether the MRI image converted by the converting unit 240 is an MRI image. In particular, the evaluation unit 260 may be used to determine whether or not the training of the converting unit 240 has been sufficiently performed. A CT image is input to the converting unit 240, and a test process is repeatedly performed by the evaluation unit 260 on the image output by the converting unit 240, for outputting the likelihood of the image output of being an MRI image and the likelihood of its being a CT image. Here, in the process of repeated tests, when the likelihood of being an MRI image continues to be higher than a predetermined value, it can be determined that the training of the converting unit 240 is sufficiently performed. The output unit 270 outputs the MRI image converted by the converting unit 240.

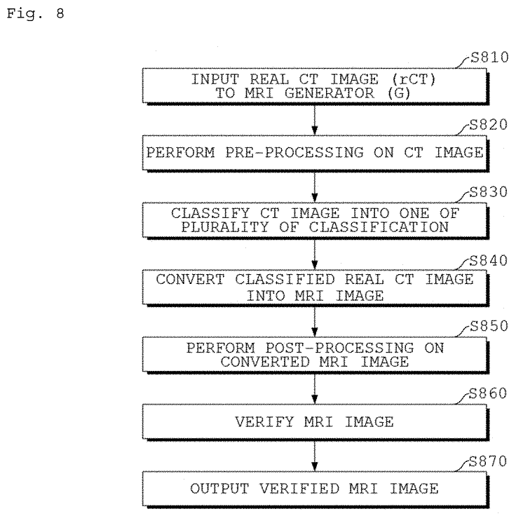

FIG. 7 is a flowchart of a training method of a converting unit of a diagnostic image converting apparatus according to at least one embodiment of the present invention.

Hereinafter, for convenience of explanation, an image taken by an MRI apparatus is referred to as a real MRI image rMRI, an MRI image generated by the MRI generator G is referred to as a converted MRI image cMRI, an image captured by a CT apparatus is referred to as a real CT image rCT, and a CT image generated by the CT generator F is referred to as a converted CT image cCT.

As described above, training of the artificial neural network of the transform unit 230 according to at least one embodiment of the present invention is a procedure for obtaining the likelihood loss and the reference loss through the forward process as shown in FIG. 5 and the backward process as shown in FIG. 6, and minimizing the likelihood loss and the reference loss by making a correction through a back propagation algorithm to weights in the plurality of arithmetic operations included in the MRU generator G, the CT generator F, the MRI discriminator MD, and the CT discriminator CD.

First, the forward process will be described with reference to FIG. 5 and FIG. 7. The converting unit 240 inputs the real CT image rCT, which is training data, to the MRI generator G in Step S710. The MRI generator G generates a converted MRI image cMRI from the real CT image rCT in Step S720. The converting unit 240 inputs the converted MRI image cMRI and the real MRI image rMRI to the MRI discriminator MD in Step S730. Then, in Step S740, the MRI discriminator MD outputs, for the converted MRI image cMRI and real MRI image rMRI each, the likelihood of each being an MRI image and the likelihood of each not being the MRI image. In Step S750, the MSL likelihood loss estimator MSL receives, from the MRI discriminator MD, the likelihood of the converted MRI image cMRI and the real MRI image rMRI each being an MRI image and the likelihood of each not being the MRI image, and calculates the likelihood losses, that is, the differences between the expected values and the output values of the likelihoods of cMRI and rMRI each being and not being an MRI image.

Meanwhile, the converting unit 240 inputs the converted MRI image cMRI output from the MRI generator G to the CT generator F in Step S760. Then, the CT generator F generates a converted CT image cCT from the converted MRI image cMRI in Step S770. In Step S780, the CT reference loss estimator CLL, then calculates a reference loss, that is, the difference between the converted CT image cCT generated by the CT generator F and the real CT image rCT input earlier in Step S710, which is training data.

Now, the backward process will be described with reference to FIGS. 6 and 7. The converting unit 240 inputs the real MRI image rMRI, which is training data, to the CT generator F in Step S715. The CT generator F generates a converted CT image cCT from the real MRI image rMRI in Step S725. The converting unit 240 inputs the converted CT image cCT and the real CT image rCT to the CT discriminator CD in Step S735. Then, in Step S745, the CT discriminator CD outputs, for the converted CT image cCT and the real CT image rCT each, the likelihood of each being a CT image and the likelihood of each not being the CT image. Then, in Step S755, the CT likelihood loss estimator CSL receives, from the CT discriminator CD, the likelihood of the converted CT image cCT and the real CT image rCT each being a CT image and the likelihood of each not being the CT image, and calculates the likelihood losses, that is, the differences between the expected values and the output values of the likelihoods of cCT and rCT each being and not being a CT image.

In Step S765, the converting unit 240 inputs the converted CT image cCT output from the CT generator F to the MRI generator G. Then, in Step S775, the MRI generator G generates a converted MRI image cMRI from the converted CT image cCT. In Step S785, the MRI reference loss estimator MLL, then calculates a reference loss, that is, the difference between the converted MRI image cMRI generated by the MRI generator G and the real MRI image rMRI input earlier in Step S715, which is training data.

Next, in Step S790, to minimize the likelihood loss and the reference loss calculated in the forward process Steps S750 and S780, and the likelihood loss and the reference loss calculated in the backward process Steps S755 and S785, a correction is made through a back propagation algorithm to weights in the plurality of arithmetic operations included in the MRI generator G, the CT generator F, the MRI discriminator MD, and the CT discriminator CD.

According to at least one embodiment of the present invention, the above-described training process is performed repeatedly by using a plurality of training data, that is, real CT images rCT and real MRI images rMRI until the likelihood losses and the reference losses are less than predetermined values. Accordingly, the converting unit 240 determines that sufficient training is completed once the forward process and the backward process described above reduced the likelihood loss and the reference loss to the predetermined value or less, when the converting unit 240 terminates the training process.

On the other hand, according to an alternative embodiment, the termination of the above-described training process may be determined by the evaluation unit 260. In other words, the evaluation unit 260 may be used to determine whether or not the training of the converting unit 240 has been sufficiently performed. Test process is repeated multiple times, wherein the evaluating unit 250 is fed with a CT image, and the evaluation unit 260 outputs the likelihood of the image output by the converting unit 240 being an MRI image, and the likelihood thereof being a CT image. Here, in the process of repeated tests, when the likelihood of being an MRI image continues to be higher than a predetermined value, it can be determined that the training of the converting unit 240 is sufficiently performed, and the training procedure may be terminated.

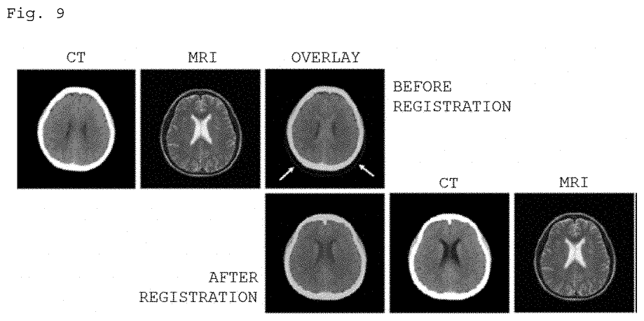

Next, a description will now be made of a method of converting a diagnostic image in accordance with at least one embodiment of the present invention. FIG. 8 is a flowchart of a diagnostic image converting method according to at least one embodiment of the present invention.

As shown in FIG. 8, when the CT image is input in Step S810, the pre-processing unit 220 performs a pre-processing on the CT image in Step S820. Here, the pre-processing includes a normalization, gray scaling, and resizing. The pre-processing in Step S820 may be omitted.

Next, in Step S830, the classifying unit 230 classifies the CT image input into one of four preset classes, and provides the classified CT image to the corresponding one of the first to fourth converting modules 241, 242, 243, and 244 of the converting unit 240. At this time, the classifying unit 230 classifies such images as taken from the top of the brain up to right before the eyeball emerges as the first layer image m1, classifies such images that range from where the eyeball emerges up to right before the lateral ventricle emerges as the second layer image m2, classifies such images that range from where the lateral ventricle emerges up to right before the ventricle disappears as the third layer image m3, and classifies such images that range from where the ventricle disappears up to the bottom of the brain as the fourth layer image m4.

Next, in Step S840, the converting unit 240 converts the CT images classified by the classifying unit 230 into an MRI image through the corresponding one of the first to fourth converting modules 241, 242, 243, and 244. Here, the corresponding converting module (any one of 241, 242, 243, and 244) includes an artificial neural network, which has been trained to convert a CT image into an MRI image, as described above with reference to FIGS. 5 to 7.

In particular, the CT image and the MRI image used as the training data of the artificial neural network of each of the first to fourth converting modules 241, 242, 243, and 244 are the corresponding layer images from among the first to fourth layer images m1, m2, m3, and m4 as described with reference to FIG. 4. Here, for both the CT image and the MRI image, the same layer image is utilized. For example, the image used for the training of the third converting module 243 is the third layer image m3 used for both the CT image and the MRI image. As described above, the brain image can be divided into a plurality of regions, so that specialized training can be performed, and a more accurate conversion result can be provided.

Subsequently, the post-processing unit 250 performs post-processing on the converted MRI image in Step S850. The post-processing may be a deconvolution to improve image quality. The post-processing of Step S850 may be omitted.

Next, in Step S860, the evaluating unit 250 verifies the MRI image converted by the converting unit 240. The evaluation unit 260 calculates the likelihood that the input image, that is, the MRI image converted by the converting unit 240 is an MRI image, and the likelihood of the MRI image converted being a CT image. Accordingly, the evaluation unit 260 determines that the verification of the image is successful when the likelihood of the MRI image converted being the MRI image is equal to or greater than the predetermined value. When the verification is successful, the evaluation unit 260 outputs the MRI image in Step S870.

FIG. 9 is an image for explaining the generation of paired data between CT and MRI images.

Ideal paired data are a pair of CT image and MRI image taken at the same time in the same part (position and structure) of the same patient, but in reality, such paired data do not exist. Therefore, a CT image and an MRI image of the same patient's position and structure at different time points can be regarded as paired data.

Even with such paired data, the CT image and the MRI image are slightly different angularly from each other in most cases, as shown in the upper part of FIG. 9, and therefore, overlaying the CT and MRI images occasionally fails to provide the desired results.

Registration between these paired data can provide the desired paired data of the CT image and the MRI image as shown at the bottom of FIG. 9.

In the example shown in FIG. 9, CT and MRI images of the same patient are aligned using affine transformation based on mutual information. As shown in FIG. 9, it can be seen that the CT and MRI images after registration are well aligned spatially and temporally.

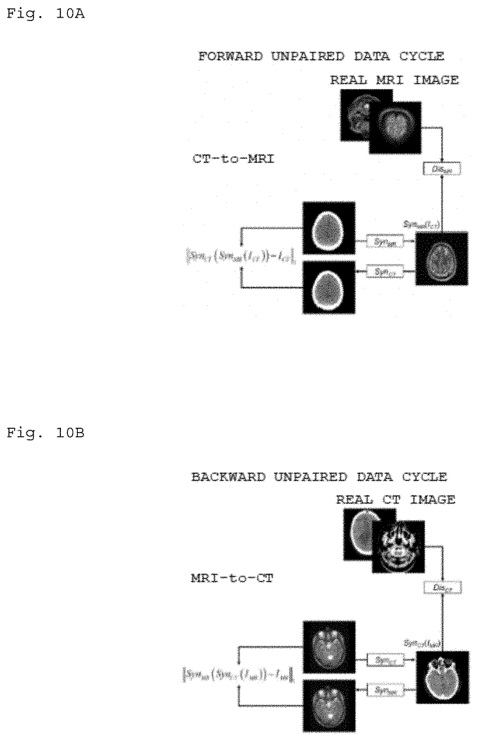

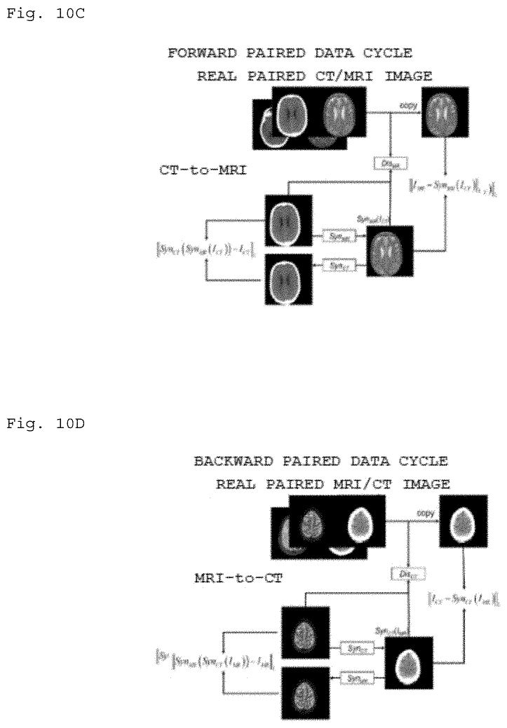

FIGS. 10A to 10D are conceptual diagrams of an example dual cycle-consistent structure using paired data and unpaired data.

In FIGS. 10A to 10D, I_{CT} represents a CT image, I_{MR} denotes an MRI image, Syn denotes a synthetic network, and Dis represents a discriminator network.

FIG. 10A shows a forward unpaired-data cycle, FIG. 10B shows a backward unpaired-data cycle, FIG. 10C shows a forward paired-data cycle, and FIG. 10D shows a backward paired-data cycle.

In the forward unpaired-data cycle, the input CT image is translated to an MRI image by a synthesis network Syn_{MR}. The synthesized MRI image is converted to a CT image that approximates the original CT image, and Dis_{MR} is trained to distinguish between real and synthesized MRI images.

In the backward unpaired-data cycle, a CT image is instead synthesized from an input MRI image by the network Syn_{CT}. Syn recomposes the MRI image from the synthesized CT image, and Dis_{CT} is trained to distinguish between real and synthesized CT images.

The forward paired-data and the backward paired-data cycle operate respectively in the same way as the above forward unpaired-data and the backward unpaired-data cycle. The difference is that Dis_{MR} and Dis_{CT} do not just discriminate between real and synthesized images, and they learn to classify between real and synthesized pairs. In addition, the voxel-wise loss between the synthesized image and the reference image is included in the paired-data cycles.

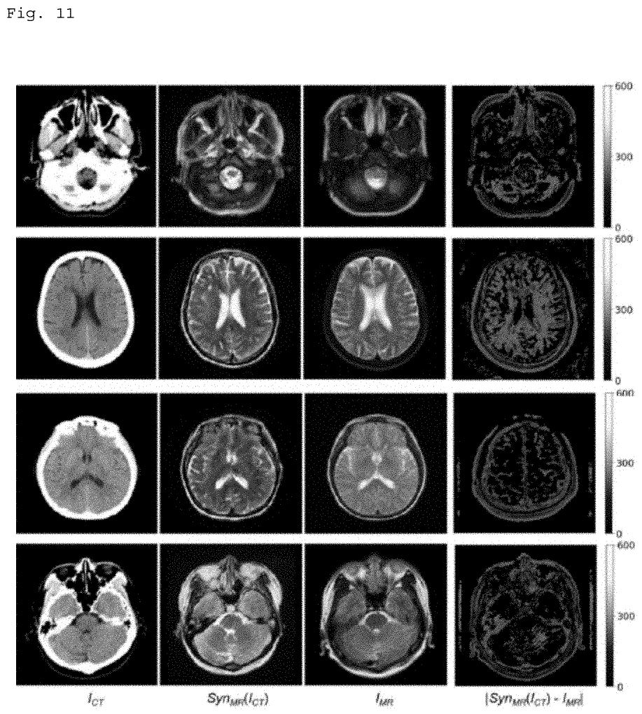

FIG. 11 is input CT images, synthesized MRI images, reference MRI images, and absolute errors between real and synthesized MRI images, when the converting of the CT images to the MRI images used the trained converting module as described above.

FIG. 11 shows from left, input CT images, synthesized MRI images, reference MRI images, and absolute errors between real and synthesized MRI images.

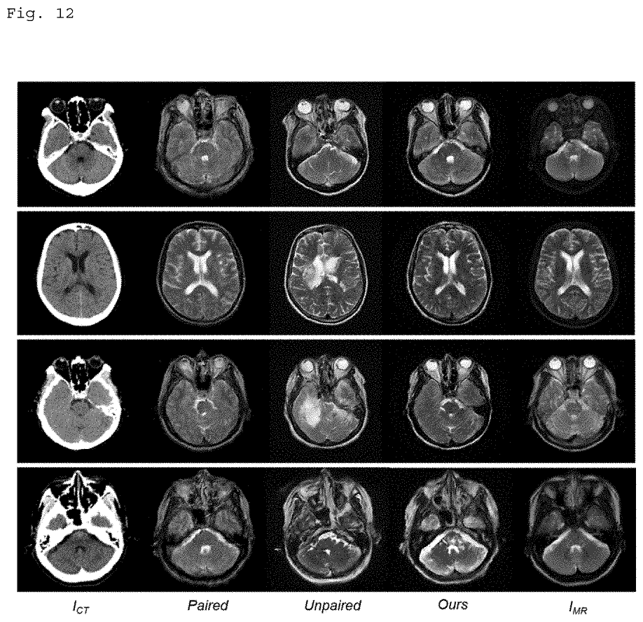

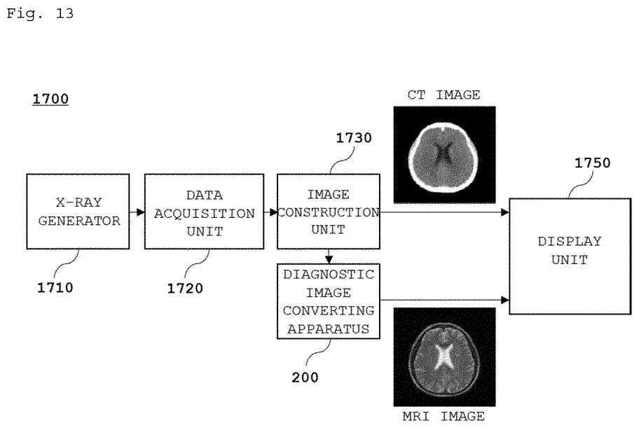

FIG. 12 is input CT images, synthesized MRI images when using paired data, unpaired data, and paired and unpaired data together, respectively, and reference MRI images.

FIG. 12 shows from left, input CT images, synthesized MRI images with paired training, synthesized MRI images with unpaired training, MRI images with paired and unpaired training, and reference MRI images.

As shown in FIG. 12, training with paired data alone exhibits a solid result, but generates blurry outputs in terms of structure. Conversely, the images obtained with unpaired data alone are realistic in terms of structure, but at the sacrifice of anatomical details.

Above all others, learning of conversion by using paired and unpaired data exhibits satisfactory results in terms of details as well as structure, as shown in the fourth column of images from the left side of FIG. 12.

FIG. 13 is a functional block diagram of a diagnostic image recording apparatus 1700 according to at least one embodiment of the present invention.

As shown in FIG. 13, the diagnostic image recording apparatus 1700 according to at least one embodiment of the present invention includes an X-ray generator 1710 for generating X-rays for CT imaging, a data acquisition unit 1720 adapted to detect the X-rays generated by the X-ray generator 1710 and penetrated a human body, to convert the detected X-rays into electrical signals, and to acquire image data from the converted electrical signals, an image construction unit 1730 for composing and outputting a CT image from the image data acquired by the data acquisition unit 1720, a diagnostic image converting apparatus 200 adapted to receive a CT image constructed by the image construction unit 1730, to convert the CT image into an MRI image, and to output the MRI image, and a display unit 1750 for displaying the CT image and the MRI image.

With the diagnostic image recording apparatus 1700, when a body part is scanned using X-rays generated from the X-ray generator 1710 according to a conventional CT imaging procedure, the image construction unit 1730 may construct a typical CT image and display the constructed CT image on the display apparatus 1750.

In addition, the diagnostic image recording apparatus 1700 inputs the CT image constructed by the image construction unit 1730 to the diagnostic image converting apparatus 200, where the CT image can be converted into the MRI image, so that the display unit 1750 can display the converted MRI image.

In at least one embodiment of the present invention, the display unit 1750 displays the CT image constructed by the image construction unit 1730 and the MRI image converted by the diagnostic image converting apparatus 200 selectively or concurrently.

As described above, the diagnostic image recording apparatus 1700 can acquire the CT image and the MRI image at the same time only by the CT imaging, thereby saving more lives in emergency situations while saving the time and cost required for the MRI imaging process.

The various methods according to at least one embodiment of the present invention described above may be implemented in a form of a program readable by various computer means and recorded in a computer-readable recording medium. Here, the recording medium may include program instructions, a data file, a data structure, or the like, alone or in combination.

The program instructions recorded on the recording medium may be those specially designed and composed for the present invention or may be available to those skilled in the art of computer software.

For example, the recording medium may be a magnetic medium such as a hard disk, a floppy disk and a magnetic tape, an optical medium such as a CD-ROM or a DVD, a magneto-optical medium such as a floptical disk, magneto-optical media, and hardware devices that are specially configured to store and execute program instructions, such as ROM, RAM, flash memory, and the like.

Examples of program instructions may include machine language wires such as those produced by a compiler, as well as high-level language wires that may be executed by a computer using an interpreter or the like. Such hardware devices may be configured to operate as one or more software modules to perform the operations of the present invention, and vice versa.

At least one embodiment of the present invention can provide a diagnostic image converting apparatus capable of obtaining an MRI image from a CT image.

At least one embodiment of the present invention can provide an apparatus for generating a diagnostic image converting module, which is capable of obtaining an MRI image from a CT image.

At least one embodiment of the present invention can provide a diagnostic image recording apparatus capable of obtaining an MRI image from a CT image.

At least one embodiment of the present invention can provide a diagnostic image converting method capable of obtaining an MRI image from a CT image.

At least one embodiment of the present invention can provide a method of generating a diagnostic image converting module capable of obtaining an MRI image from a CT image.

At least one embodiment of the present invention can provide a diagnostic image recording method capable of obtaining an MRI image from a CT image.

According to at least one embodiment of the present invention, the CT image can be converted into an MRI image, thereby saving more time and cost for MRI imaging as well as saving more lives in emergency situations.

Although exemplary embodiments of the present invent ion have been described for illustrative purposes, those skilled in the art will appreciate that various modifications, additions and substitutions are possible, without departing from the idea and scope of the claimed invention. Accordingly, one of ordinary skill would understand the scope of the claimed invention is not to be limited by the explicitly described above embodiments but by the claims and equivalents thereof.

1. An apparatus for converting a diagnostic image, the apparatus comprising:
an input unit for inputting a CT image;
a converting module configured to convert the CT image inputted via the input unit into an Mill image; and
an output unit configured to output the MRI image converted by the converting module.

2. The apparatus according to claim 1, further comprising a classifying unit configured to classify the CT image inputted via the input unit by positions of recorded tomographic layers, wherein
the converting module is configured to convert the CT image classified by the classifying unit into the MRI image.

3. The apparatus according to claim 2, wherein the classifying unit is configured, by the positions of the recorded tomographic layers,
to classify an image of from a top of a brain to right before an eyeball appears as a first layer image,
to classify an image of from the eyeball beginning to appear to right before a lateral ventricle appears as a second layer image,
to classify an image of from the lateral ventricle beginning to appear to right before a ventricle disappears as a third layer image, and
to classify an image of from the ventricle disappears to a bottom of the brain as a fourth layer image.

4. The apparatus according to claim 3, wherein the converting module includes
a first converting module configured to convert a CT image classified as the first layer image into the MRI image,
a second converting module configured to convert a CT image classified as the second layer image into the MRI image,
a third converting module configured to convert a CT image classified as the third layer image into the MRI image, and
a fourth converting module configured to convert a CT image classified as the fourth layer image into the MRI image.

5. The apparatus according to claim 1, further comprising a pre-processing unit configured to perform a pre-processing including at least one of normalization, gray scaling, or resizing on the CT image inputted via the input unit.

6. The apparatus according to claim 1, further comprising a post-processing unit configured to perform a post-processing including a deconvolution on the MRI image converted by the converting module.

7. The apparatus according to claim 1, further comprising an evaluation unit configured to output a first likelihood that the MRI image converted by the converting module is a CT image and a second likelihood that the MRI image converted by the converting module is an MRI image.

8. An apparatus for generating a converting module of the apparatus for converting a diagnostic image according to claim 1, the apparatus comprising:
an MRI generator configured, when a first CT image that is training data is inputted, to generate a first MRI image from the first CT image by performing a plurality of operations;
a CT generator configured, when a second MRI image that is training data is inputted, to generate a second CT image from the second MRI image by performing a plurality of operations;
an MRI discriminator configured, when the first MRI image and the second MRI image are inputted, to output a first likelihood of the input image being an MRI image and a second likelihood of the input image not being an MRI image by performing a plurality of operations;
a CT discriminator configured, when the first CT image and the second CT image are inputted, to output a third likelihood of the input image being a CT image and a fourth likelihood of the CT image not being a CT image by perform a plurality of operations;
an MRI likelihood loss estimator configured to calculate a first likelihood loss that is a difference between an expected value and an output value of the first likelihood and the second likelihood outputted from the Mill discriminator;
a CT likelihood loss estimator configured to calculate a second likelihood loss that is a difference between an expected value and an output value of the third likelihood and the fourth likelihood outputted from the CT discriminator;
an MRI reference loss estimator configured to calculate a first reference loss that is a difference between the first Mill image and the second MRI image; and
a CT reference loss estimator configured to calculate a second reference loss that is a difference between the first CT image and the second CT image, wherein
the apparatus is configured to adjust weights included in the plurality of operations performed by the Mill generator, the CT generator, the Mill discriminator, and the CT discriminator using a back propagation algorithm, in order to minimize the first and second likelihood losses and the first and second reference losses.

9. The apparatus according to claim 8, wherein the apparatus is configured to adjust the weights by using paired data and unpaired data.

10. An apparatus for recording a diagnostic image, the apparatus comprising:
an X-ray generator configured to generate X-rays for CT imaging;
a data acquisition unit configured to detect the X-rays generated by the X-ray generator and penetrated through a human body, to convert detected X-rays into electrical signals, and to acquire image data from converted electrical signals;
an image construction unit configured to construct a CT image from the image data acquired by the data acquisition unit and to output the CT image;
the apparatus for converting a diagnostic image according to claim 1, configured to receive the CT image constructed by the image construction unit, to convert the CT image into an MRI image, and to output the MRI image; and
a display unit configured to display the CT image and the MRI image selectively or concurrently.

11. A method of converting a diagnostic image, the comprising:
inputting a CT image;
converting the CT image inputted at the inputting into an Mill image; and
outputting the Mill image converted at the converting.

12-17. (canceled)

18. A method of generating a converting module used at the converting in the method of converting a diagnostic image according to claim 11, the method comprising:
first generating including generating, when a first CT image that is training data is inputted, a first Mill image from the first CT image by performing a plurality of operations;
second generating including generating, when a second MRI image that is training data is inputted, a second CT image from the second MRI image by performing a plurality of operations;
first outputting including outputting, when the first MRI image and the second MRI image are inputted, a first likelihood of the input image being an MRI image and a second likelihood of the input image not being an Mill image by performing a plurality of operations;
second outputting including outputting, when the first CT image and the second CT image are inputted, a third likelihood of the input image being a CT image and a fourth likelihood of the CT image not being a CT image by perform a plurality of operations;
calculating a first likelihood loss that is a difference between an expected value and an output value of the first likelihood and the second likelihood outputted at the first outputting;
calculating a second likelihood loss that is a difference between an expected value and an output value of the third likelihood and the fourth likelihood outputted at the second outputting;
calculating a first reference loss that is a difference between the first MRI image and the second MRI image;
calculating a second reference loss that is a difference between the first CT image and the second CT image; and
adjusting weights included in the plurality of operations performed at the first generating, the second generating, the first outputting, and the second outputting using a back propagation algorithm, in order to minimize the first and second likelihood losses and the first and second reference losses.

19. (canceled)

20. A method of recording diagnostic image, the method comprising:
generating X-rays for CT imaging;
acquiring including detecting the X-rays generated at the generating and penetrated through a human body, converting detected X-rays into electrical signals, and acquiring image data from converted electrical signals;
first outputting including constructing a CT image from the image data acquired at the acquiring and outputting the CT image;
converting including performing the method of converting a diagnostic image according to claim 11, by receiving the CT image constructed at the constructing, converting the CT image into an Mill image, and outputting the MRI image; and
displaying the CT image and the Mill image selectively or concurrently.

21. A non-transitory computer readable recording medium storing a computer program including computer-executable instructions for causing, when executed by a processor, the processor to perform the method of converting a diagnostic image according to claim 11.

22. A non-transitory computer readable recording medium storing a computer program including computer-executable instructions for causing, when executed by a processor, the processor to perform the method of generating a converting module according to claim 18.

23. A non-transitory computer readable recording medium storing a computer program including computer-executable instructions for causing, when executed by a processor, the processor to perform the method of recording a diagnostic image according to claim 20.
